(19) United States
(12) Patent Application Publication
(10) Pub. No.: US 2019/0209119 A1
(43) Pub. Date: Jul. 11, 2019

(54) System and Method for Angular Alignment of a Probe at a Target Location

(71) Applicant: Rivanna Medical LLC, Charlottesville, VA (US)

(72) Inventors: Frank William Mauldin, JR., Charlottesville, VA (US); Adam Dixon, Charlottesville, VA (US); Kevin Owen, Crozet, VA (US)

(21) Appl. No.: 15/864,395

(22) Filed: Jan. 8, 2018

(51) Int. Cl. A61B 8/08; A61B 8/00; A61B 5/06

(52) U.S. Cl. CPC A61B 8/0841; A61B 8/5223; A61B 5/061; A61B 8/465

(57) ABSTRACT

An ultrasound imaging device includes an angle sensor to measure the angular orientation of the device. A first user input causes the device to store the first angular orientation in memory as a stored angular orientation. A second user input causes the device to determine a second angular orientation of the device or of a projected path of a probe through a probe guide. The device compares the second angular orientation with the first angular orientation and provides a visual indication of the direction(s) to rotate the device and/or probe holder and/or probe so that the second angular orientation is equal to or substantially equal to the stored angular orientation.

### TECHNICAL FIELD

This application relates generally to locating target regions in ultrasound imaging applications.

### BACKGROUND

Medical ultrasound is commonly used to facilitate needle injection or probe insertion procedures such as central venous line placement or various spinal anesthesia procedures. A commonly implemented technique involves locating anatomical landmarks (e.g. blood vessel or bone structures) using ultrasound imaging and subsequently marking the patient's skin with a surgical marker in proximity to the ultrasound transducer. The ultrasound transducer is then removed, and the needle is inserted after positioning the needle at a location relative to the marking sites.

Current ultrasound devices do not have a mechanism to determine whether the angular orientation of the needle is the same as or substantially the same as the angular orientation of the ultrasound device when it located the anatomical landmark through ultrasound imaging. If the needle is not at same or substantially the same angular orientation as the ultrasound device when it located the anatomical landmark through ultrasound imaging, the needle may miss the anatomical landmark even though it is inserted at the marked position on the patient's skin. It would be desirable to overcome this and other deficiencies in existing systems and methods.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

An aspect of the invention is direct to a handheld ultrasound imaging device comprising a housing; an ultrasound imaging unit disposed in the housing proximal to a first surface of the housing; an angle sensor disposed in the housing, the angle sensor outputting an angular orientation signal, the angular orientation signal corresponding to a measured angular orientation of the housing; a display disposed on a second surface of the housing, the first and second surfaces opposing one another, the display including a graphical user interface; said graphical user interface including a first graphical user interface element that generates a first GUI output signal in response to a first user input and a second graphical user interface element that generates a second GUI output signal in response to a second user input; a memory disposed in the housing; and a processor disposed in the housing, the processor in electrical communication with the display and the angle sensor, wherein a receipt of the first GUI output signal causes the processor to store the measured angular orientation in the memory as a stored angular orientation and a receipt of the second GUI output signal causes the processor to compare the stored angular orientation with a current value of the measured angular orientation.

In one or more embodiments, the receipt of the second GUI output signal causes the processor to calculate a direction to rotate the housing to align the current value of the measured angular orientation with the stored angular orientation. In one or more embodiments, the device further comprises a probe guide coupled to the housing, the probe guide having a predetermined path along which to insert a probe. In one or more embodiments, the device further comprises a marking unit coupled to the housing and configured to produce a mark on a surface of a target to be imaged.

In one or more embodiments, the processor is configured to generate a direction output signal that causes the display to graphically indicate, on the graphical user interface, an indication of the current angular orientation relative to the stored angular orientation. In one or more embodiments, the direction output signal causes the display to graphically indicate, on the graphical user interface, a direction to rotate the probe guide. In one or more embodiments, the direction output signal causes the display to graphically indicate a first direction to rotate the probe guide to adjust an elevation angle of a projected probe path of the probe. In one or more embodiments, the direction output signal causes the display to graphically indicate a second direction to rotate the probe guide to adjust an azimuthal angle of a projected probe path of the probe. In one or more embodiments, the direction output signal causes the display to graphically indicate a first direction to rotate the probe guide to adjust an elevation angle of the projected probe path of the probe such that the display simultaneously graphically indicates the first and second directions to rotate the probe guide.

In one or more embodiments, the processor is configured to generate an alignment signal when the current value of the measured angular orientation is substantially equal to the stored angular orientation. In one or more embodiments, the alignment signal causes the display to graphically indicate, on the graphical user interface, that the current value of the measured angular orientation is substantially equal to the stored angular orientation. In one or more embodiments, the alignment signal causes a speaker to generate an audible alert to indicate that the current value of the measured angular orientation is substantially equal to the stored angular orientation, the speaker disposed on or in the housing. In one or more embodiments, the alignment signal causes an LED to emit a light to indicate that the current value of the measured angular orientation is substantially equal to the stored angular orientation, the LED disposed on the housing. In one or more embodiments, the angle sensor includes an accelerometer or a gyroscope.

Another aspect of the invention is directed to an ultrasound imaging method comprising, in a probe guidance system comprising a processor disposed in a housing: determining a measured angular orientation of the housing with an angle sensor disposed in the housing; receiving a first user input to store the measured angular orientation; in response to the first user input, storing the measured angular orientation as a stored angular orientation in a memory in electrical communication with the processor; receiving a second user input to determine an angular alignment of a projected probe path of a probe disposed in the probe holder, the probe holder coupled to the housing; and in response to the second user input, comparing a current measured angular orientation of the projected probe path with the stored angular orientation.

In one or more embodiments, the method further comprises generating a visual indication of a direction to rotate the probe holder to align the current measured angular orientation with the stored angular orientation. In one or more embodiments, the method further comprises transmitting one or more ultrasound signals from one or more transducers in the probe guidance system; obtaining ultrasound data generated based, at least in part, on one or more ultrasound signals from an imaged region of a subject; and displaying an ultrasound image of a target anatomy in the subject based, at least in part, on the ultrasound data.

In one or more embodiments, the method further comprises adjusting an angular orientation of the housing to align the housing with a target location in the target anatomy; and marking a skin surface of the subject corresponding to the target location in the target anatomy. In one or more embodiments, the target anatomy comprises a spine and the target location comprises an epidural space in the spine. In one or more embodiments, the method further comprises aligning the projected probe path of the probe with the mark on the skin surface; and adjusting the angular orientation of the probe holder according to the visual indication so the current measured angular orientation is substantially aligned with the stored angular orientation. In one or more embodiments, the method further comprises inserting the probe into the skin surface along the projected probe path, the probe passing through the mark on the skin surface while the current measured angular orientation is substantially aligned with the stored angular orientation.

In one or more embodiments, the visual indication indicates a first direction to rotate the probe guide to adjust an elevation angle of the projected probe path. In one or more embodiments, the visual indication indicates a second direction to rotate the probe guide to adjust an azimuthal angle of the projected probe path. In one or more embodiments, the visual indication indicates a first direction to rotate the probe guide to adjust an elevation angle of the projected probe path such that the visual indication simultaneously indicates the first and second directions to rotate the probe guide. In one or more embodiments, the method further comprises generating a visual alignment indication that indicates that the current measured angular orientation is substantially aligned with the stored angular orientation.

### BRIEF DESCRIPTION OF THE DRAWINGS

For a fuller understanding of the nature and advantages of the present concepts, reference is made to the following detailed description of preferred embodiments and in connection with the accompanying drawings, in which:

FIG. 1 is a block diagram of an ultrasound imaging device according to one or more embodiments;

FIG. 2 is a side view of an alignment of a first elevation angle of an ultrasound imaging device with a target anatomical feature according to one or more embodiments;

FIG. 3 is a top view of an alignment of a first azimuthal angle of an ultrasound imaging device with a target anatomical feature according to one or more embodiments;

FIG. 4 is a side view of a misalignment of a second elevation angle of with respect to the first elevation angle according to one or more embodiments;

FIG. 5 is a top view of a misalignment of a second azimuthal angle with respect to the first azimuthal angle according to one or more embodiments;

FIG. 6 is a front view of a display that indicates the directions to rotate the device to align the second elevation and azimuthal angles with the first elevation and azimuthal angles, respectively, according to one or more embodiments;

FIG. 7 is a flow chart of a method for aligning an orientation of a probe according to one or more embodiments;

FIG. 8 is a side view of a misalignment of the second elevation angle with respect to the first elevation angle according to an alternative embodiment; and

FIG. 9 is a top view of a misalignment of a second azimuthal angle with respect to the first azimuthal angle according to an alternative embodiment.

### DETAILED DESCRIPTION

An ultrasound imaging device includes an angle sensor that measures the angular orientation (e.g., elevation angle and/or azimuthal angle) of the device or of the ultrasound transducer component of the device. The device is used to locate a target location on an anatomical landmark in a subject through ultrasound imaging. When the target location is identified, the position on the subject's skin is marked and the angular orientation of the device is stored, as the first angular orientation, in a memory disposed in or operatively coupled to the device. At a later point in time, a probe is desired to be inserted through the marked position on the patient's skin to the target location. At this point, a probe guide is placed above the patient's skin such that the projected path of the probe passes through the marked position on the patient's skin. In this position and orientation, the angular orientation of the probe guide is determined and compared to the first angular orientation. The angular orientation of the probe holder is then adjusted until it is the same as or substantially the same as the first angular orientation.

In some embodiments, the ultrasound imaging device can include the probe guide, in which case the angular orientation of the probe guide and the device (e.g., the housing of the device) are adjusted to match or substantially match the first angular orientation. When the angular orientation of the probe guide matches or substantially matches the first angular orientation, the probe can be inserted into the subject through the marked surface on the subject's skin.

In other embodiments, the probe guide is a separate component, and the probe guide and the device are held so that the angular orientation of the device is the same as or substantially the same as the angular orientation of the probe guide. This allows the angle sensor in the device to indirectly measure the angular orientation of the probe guide. The angular orientation of the probe guide and the device can then be adjusted so that their angular orientation is the same as or substantially the same as the first angular orientation. When the angular orientation of the probe guide matches or substantially matches the first angular orientation, the probe can be inserted into the subject through the marked surface on the subject's skin.

In an alternative embodiment, there is no probe holder guide and the probe itself and the device are held so that the angular orientation of the device is the same as or substantially the same as the angular orientation of the probe. This allows the angle sensor in the device to indirectly measure the angular orientation of the probe. The angular orientation of the probe and the device can then be adjusted so that their angular orientation is the same as or substantially the same as the first angular orientation. When the angular orientation of the probe matches or substantially matches the first angular orientation, the probe can be inserted into the subject through the marked surface on the subject's skin.

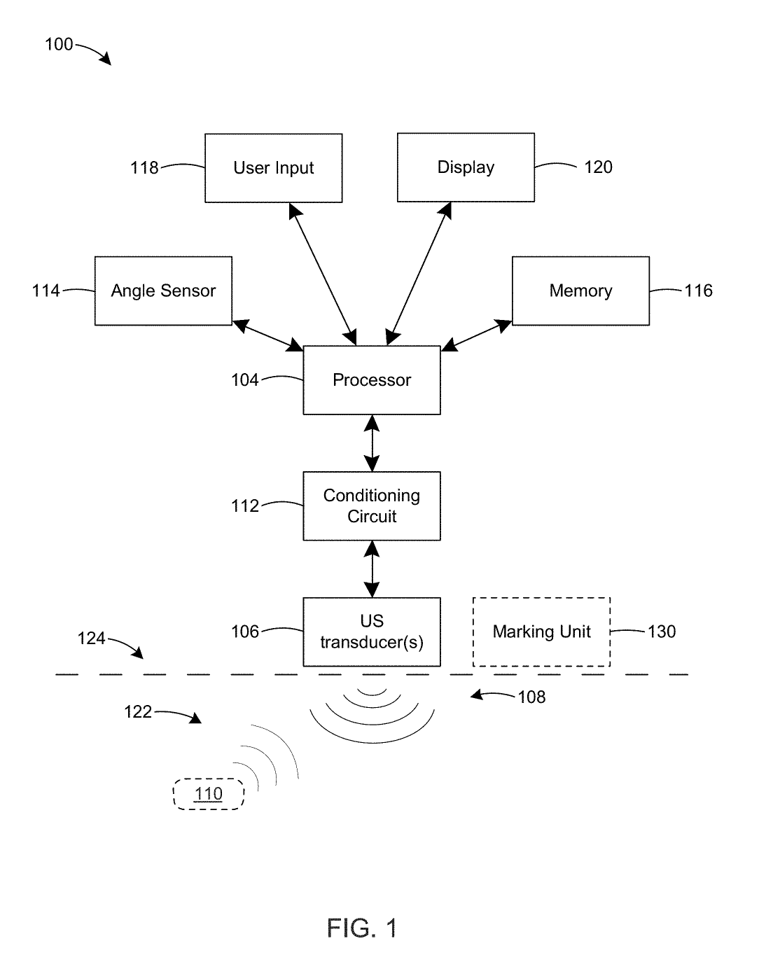

FIG. 1 is a block diagram of an ultrasound imaging device 100 according to one or more embodiments. As shown, device 100 comprises a processor 104, one or more ultrasound transducers 106, an ultrasound signal conditioning circuit 112, an angle sensor 114, a memory 116, a user input 118, a display 120, and an optional marking unit 130. The one or more ultrasound transducers 106 are configured to generate ultrasonic energy 108 to be directed at a target anatomical structure 110 within a subject being imaged (e.g., the ultrasound transducer(s) 106 may be configured to insonify one or more regions of interest within the subject).

Some of the ultrasonic energy 108 may be reflected by the target anatomical structure 110, and at least some of the reflected ultrasonic energy 122 may be received by the ultrasound transducers 106. In some embodiments, the at least one ultrasonic transducer 106 may form a portion of an ultrasonic transducer array, which may be placed in contact with a surface 124 (e.g., skin) of a subject being imaged. In some embodiments, ultrasonic energy reflected 120 by the subject being imaged may be received by ultrasonic transducer(s) 106 and/or by one or more other ultrasonic transducers, such as one or more ultrasonic transducers that are part of a transducer array. The ultrasonic transducer(s) that receive the reflected ultrasonic energy may be geometrically arranged in any suitable manner (e.g., as an annular array, a piston array, a linear array, a two-dimensional array, or other array or geometrical arrangement) or in any other suitable way, as aspects of the disclosure provided herein are not limited in this respect.

As illustrated in FIG. 1, ultrasonic transducer(s) 106 are coupled to the ultrasonic signal conditioning circuit 112, which is shown as being coupled to processor 104 in device 100. The ultrasonic signal conditioning circuit 112 may include various types of circuitry for use in connection with ultrasound imaging such as beam-forming circuitry, for example. As other examples, the ultrasonic signal conditioning circuit may comprise circuitry configured to amplify, phase-shift, time-gate, filter, and/or otherwise condition received ultrasonic information (e.g., echo information), such as provided to the processor 104.

In some embodiments, the receive path from each transducer element from part of a transducer array, such as an array including the ultrasonic transducer(s) 106, may include one or more of a low noise amplifier, a main-stage amplifier, a band-pass filter, a low-pass filter, and an analog-to-digital converter. In some embodiments, one or more signal conditioning steps may be performed digitally, for example by using the processor 104.

The ultrasound imaging apparatus may include one or more ultrasound transducers 106 configured to obtain depth information via reflections of ultrasonic energy from an echogenic target anatomical structure 110, which may be a bone target, blood vessel, lesion, or other anatomical target. In some embodiments, the device 100 can be configured to obtain ultrasonic echo information corresponding to one or more planes perpendicular to the surface of an array of ultrasound transducer(s) 106 (e.g., to provide “B-mode” imaging information). In addition or in the alternative, the device 100 can be configured to obtain information corresponding to one or more planes parallel to the surface of an array of ultrasound transducer(s) 106 (e.g., to provide a “C-mode” ultrasound image of loci in a plane parallel to the surface of the transducer array at a specified depth within the tissue of the subject). In an example where more than one plane is collected, a three-dimensional set of ultrasonic echo information can be collected.

The processor 104 is coupled to memory 116, which can include one or more non-transitory computer-readable media, such as RAM, ROM, a disk, and/or one or more other memory technology or storage devices. Computer-readable instructions for operating the device 100 can be stored on memory 116. The processor 104 can also store information in memory 116, such as the angle of device 100 measured by angle sensor 114.

The processor controller circuit 104 (or one or more other processor circuits) is communicatively coupled to user input device 118. User input device 118 can include one or more of a keypad, a keyboard (e.g., located near or on a portion of ultrasound scanning assembly, or included as a portion of a workstation configured to present or manipulate ultrasound imaging information), a mouse, a rotary control (e.g., a knob or rotary encoder), one or more physical buttons, one or more virtual buttons displayed on display 120 (e.g., in a graphical user interface on display 120), a soft-key touchscreen aligned with or displayed on display 120 (e.g., in a graphical user interface on display 120), and/or one or more other controls or user input devices of any suitable type.

In some embodiments, the processor controller circuit 104 may be configured to perform model registration-based imaging and presenting the constructed image or images to the user via the display 120. For example, a simultaneous 2D/3D display may be presented to the user via the display 120. An example of a commercially-available model registration-based imaging system is SpineNav3d™, available from Rivanna Medical, LLC. Additional details of a model registration-based imaging system, according to some embodiments, are described in U.S. Patent Application Publication No. 2016/0012582, titled “Systems and Methods of Ultrasound Imaging.” Using these or other methods known to those skilled in the art, certain anatomical image targets can be automatically identified.

The device 100 includes an optional marking unit 130 that is configured to indicate proper placement of a probe (e.g., a needle and/or a catheter) along the surface 124 of the target to be imaged, in some embodiments. In certain embodiments, the marking unit 130 is configured to identify a target surface 124 location (e.g., an insertion location) corresponding to a center of an imaging scan plane. The marking unit 130 can comprise, in certain embodiments, a probe indicator configured to indicate proper placement of a probe at or near a target that is to be imaged. For example, in some embodiments, the marking unit 130 comprises an identifying mark indicating the target surface 124 location. The identifying mark can comprise, for example, a hole, an indentation, an ink dot, or other identifying mark. The marking unit 130 can be detachable in some embodiments.

In some embodiments, ultrasonic energy reflected 122 from target anatomical 110 may be obtained or sampled after signal conditioning through the ultrasound signal conditional circuit 112 as the device 100 is rotated. The angle of device 100 (e.g., the elevation angle and/or the azimuthal angle) can be measured by angle sensor 114. Angle sensor 114 can be any suitable type of sensor configured to obtain information about the absolute or relative angle of device 100. For example, the angle sensor 114 can include an accelerometer (e.g., configured to sense gravitational acceleration along one or more axes), a gyroscope, an angular position sensor circuit, an optical sensor, and/or other angle sensing technology.

Angle information from the angle sensor 114 may be sent to the processor 104, which can act on the angle information based on user input from user input device 118. For example, a first user input (e.g., pressing a first button) can cause the processor 104 to store the current value, at the time of the first user input, of the angle information in memory 116. The angle information can include the measured angular orientation of device 100, such as the elevation angle and/or the azimuthal angle of the housing of device 100. In some embodiments, the user provides the first user input when the device 100 is aligned with an anatomical feature of the target anatomical structure 110, such as the epidural space of the spinal cord (e.g., prior to epidural anesthesia), at which point the user can mark the position of the device 100 on surface 124 with marking unit 130.

A second user input (e.g., pressing a second button) can cause the processor 104 to compare the current value, at the time of the second user input, of the angle information with the stored angle information in memory 116 (i.e., the angle information stored in response to the first user input). In some embodiments, the user provides the second user input when he/she is ready to begin the anesthesia procedure. For example, after the first user input, the user can prepare the skin surface 124 for the anesthesia procedure (e.g., by applying an antiseptic agent) and then can align a probe with the marked position on the skin surface 124. The probe can be disposed in a probe guide, which can be attached to or integrally connected to device 100 or which can be a separate unit. In some embodiments, the angle information can include the measured angular orientation of the probe guide, which can be parallel to the measured angular orientation of the device 100, such as the elevation angle and/or the azimuthal angle of the housing of device 100.

If the current value, at the time of the second user input, of the angle information is equal to or approximately equal to (e.g., within 5% or 10%) the stored angle information in memory 116, the processor 104 generates an alignment signal to alert the user that the device 100 and/or the projected probe path is currently aligned with the angular orientation of the device 100 at the time of the first user input. The alignment signal can cause display 120 to display a visual image that indicates that the device 100 and/or the projected probe path is aligned. In addition or in the alternative, the alignment signal can cause the device 100 to generate an audible sound (e.g., through a speaker in electrical communication with processor 104), a light (e.g., an LED) to emit light at a particular frequency, and/or other signal to the user.

If the current value, at the time of the second user input, of the angle information is not equal to or approximately equal to (e.g., within 5% or 10%) the stored angle information in memory 116, the processor 104 generates a misalignment signal to alert the user that the device 100 and/or the projected probe path is not currently aligned with the angular orientation of the device 100 at the time of the first user input. The misalignment signal can include a direction output signal that causes the display 120 to graphically indicate one or more directions to rotate the device 100 and/or the probe holder so that it is aligned with the angular orientation of the device 100 at the time of the first user input. The direction output signal can indicate a first direction to rotate the device 100 and/or probe holder to adjust an elevation angle of the device 100 and/or probe holder. In addition or in the alternative, the direction output signal can indicate a second direction to rotate the device 100 and/or probe holder to adjust an azimuthal angle of the device 100 and/or probe holder. In some embodiments, the display 120 can simultaneously display an indication to rotate the device 100 and/or probe holder in the first and the second directions to adjust its elevation and azimuthal angles, respectively. The misalignment signal can also cause the device 100 to generate an audible sound (e.g., through a speaker in electrical communication with processor 104), a light (e.g., an LED) to emit light at a particular frequency, and/or other signal to the user. The sounds, light, and/or other signal generated in response to the alignment signal can be different than the sounds, light, and/or other signal generated in response to the misalignment signal.

In some embodiments, device 100 may provide imaging using non-ionizing energy, it may be safe, portable, low cost, and may provide an apparatus or technique to align an insertion angle of a probe to reach a desired target depth or anatomical location. Examples of the apparatus and methods described herein are described in the context of imaging the spinal bone anatomy. However, the apparatus and methods described herein are not limited to being used for imaging of the spine and may be used to image any suitable target anatomy such as bone joints, blood vessels, nerve bundles, nodules, cysts, or lesions.

It should be appreciated that the device 100 described with reference to FIG. 1 is an illustrative and non-limiting example of an apparatus configured to perform ultrasound imaging in accordance with embodiments of the disclosure provided herein. Many variations of device 100 are possible. For example, in some embodiments, an ultrasound imaging apparatus may comprise one or more transducers for generating ultrasonic energy and circuitry to receive and process energy reflected by a target being imaged to generate one or more ultrasound images of the subject, but may not comprise a display to display the images. Instead, in some embodiments, an ultrasound imaging apparatus may be configured to generate one or more ultrasound images and may be coupled to one or more external displays to present the generated ultrasound images to one or more users.

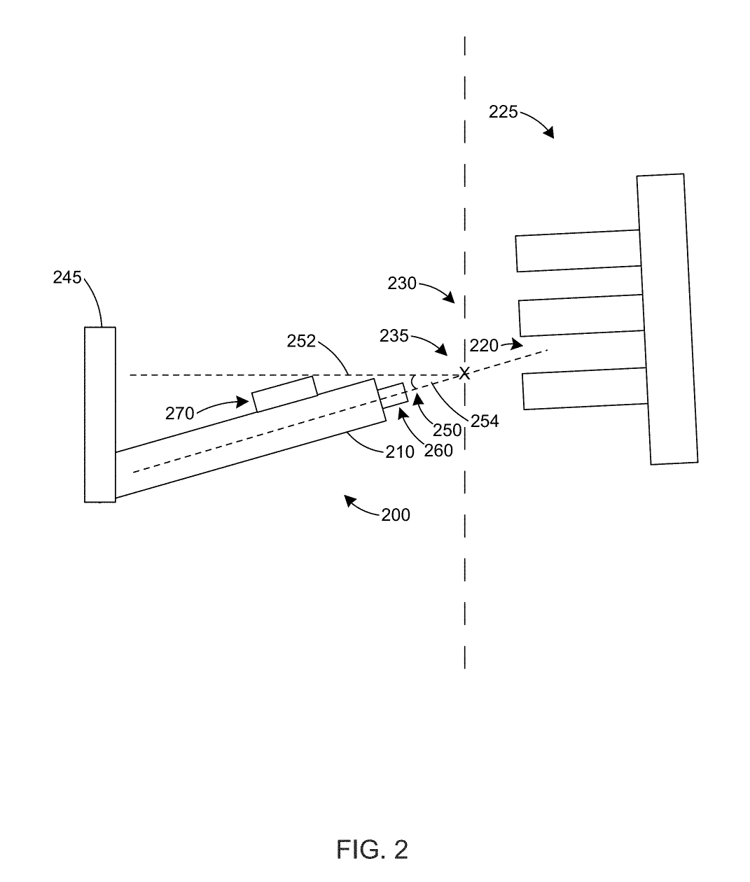

FIG. 2 is a side view of an alignment of a first elevation angle 250 of an ultrasound imaging device 200 with a target anatomical feature according to one or more embodiments. The ultrasound imaging device 200 can be include the same or substantially the same components as device 100 described above. In operation, a user aligns the angular orientation and position of housing 210 of the device 200 with the epidural space 220 of a spine 225 of a subject using ultrasound images displayed on display 245. The user then marks 235 the skin surface 230 that corresponds to the epidural space 220 with a marking unit 260 on device 200 while the device 200 is in the aligned angular orientation and position. The marking unit 260 can include a laser, a lancing unit, a mechanical indentation unit, a needle, or a material deposition member (e.g., a pen or a marker). In other embodiments, the marking unit 260 can include a detachable template that includes an identifying mark such as a hole, an indentation, or other identifying mark. In some embodiments, the marking unit 260 is the same as or similar to one or more of the marking units described in U.S. Pat. No. 9,486,291 and U.S. Patent Application Publication No. 2016/0007956, which are hereby incorporated by reference. Ultrasound imaging device 200 also includes an optional probe guide 270 that has a channel through which to guide the insertion of a probe (e.g., a needle) into the subject. The probe guide 270 can be detachable from or it can be integrated into the housing 210 of device 400.

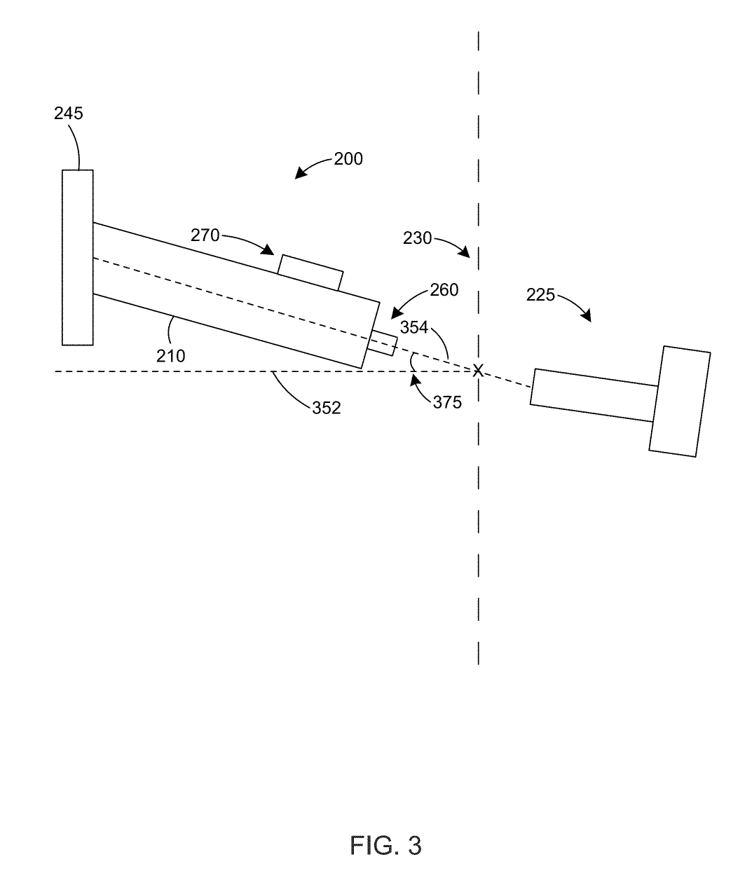

The first elevation angle 250 is defined by a normal 252 to the skin surface 230 that passes through the marking 235 and a line 254 that passes through the center of housing 210, the marking 235, and the desired portion of epidural space 220. The first azimuthal angle 375 is defined by a normal 352 to the skin surface 230 that passes through the marking 235 and a line 354 that passes through the center of housing 210 and the marking 235, as illustrated in FIG. 3.

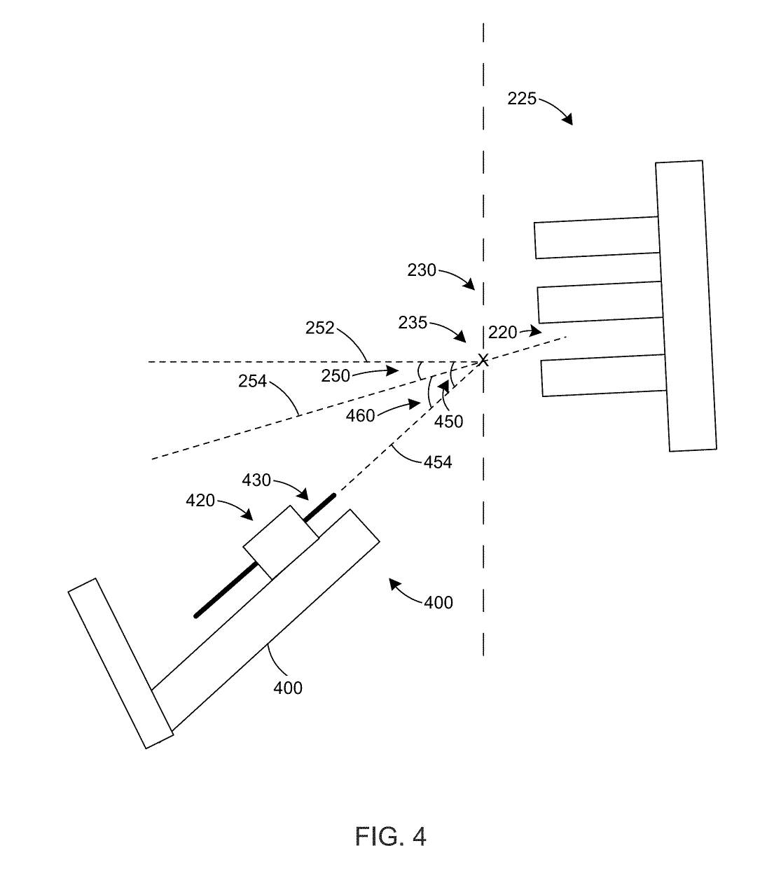

FIG. 4 is a side view of a misalignment of a second elevation angle 450 of with respect to the first elevation angle 250 according to one or more embodiments. Ultrasound imaging device 400 can be the same as, substantially the same as, or different than device 200. Device 400 includes an optional probe guide or holder 420 that has a channel through which to guide the insertion of a probe 430 (e.g., a needle) into a subject. The probe guide 420 can be detachable from or it can be integrated into the housing 410 of device 400. After the skin surface 230 proximal to marking 235 is prepped (e.g., disinfected, etc.) for the procedure that involves the probe 430, the user holds or places the device 400 next to the subject so that the projected path 454 of the probe 430 passes through marking 235.

In this position and orientation, the second elevation angle 450 is defined by a normal 252 to the skin surface 230 that passes through the marking 235 and the projected path 454 of probe 430. The processor (e.g., processor 104) in device 400 compares the second elevation angle 450 with the first elevation angle 250 to determine if they are aligned. In this case, the processor determines that the second elevation angle 450 is not equal to or substantially equal to the first elevation angle 250. The processor calculates the elevation angle error 460 as the difference between the second elevation angle 450 and the first elevation angle 250. The processor also determines the direction for the user to rotate the device 200 and/or the probe holder 420 and/or the probe 430 to reduce the elevation angle error 460 so that the second elevation angle 450 will be equal to or substantially equal to the first elevation angle 250.

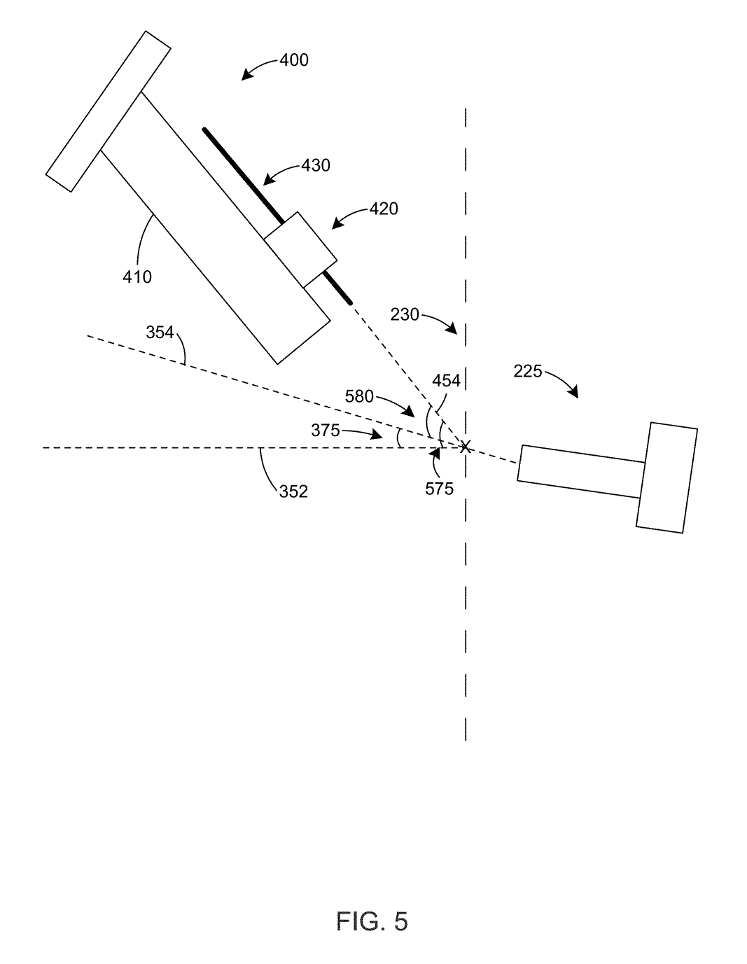

FIG. 5 is a top view of a misalignment of a second azimuthal angle 575 of with respect to the first azimuthal angle 250 according to one or more embodiments. The second azimuthal angle 575 is defined by a normal 352 to the skin surface 230 that passes through the marking 235 and the projected path 454 of probe 430. The processor (e.g., processor 104) in device 400 compares the second azimuthal angle 575 with the first azimuthal angle 375 to determine if they are aligned. In this case, the processor determines that the second azimuthal angle 575 is not equal to or substantially equal to the first azimuthal angle 375. The processor calculates the azimuthal angle error 580 as the difference between the second azimuthal angle 575 and the first azimuthal angle 375. The processor also determines the direction for the user to rotate the device 400 and/or the probe holder 420 and/or the probe 430 to reduce the azimuthal angle error 580 so that the second azimuthal angle 575 will be equal to or substantially equal to the first azimuthal angle 375.

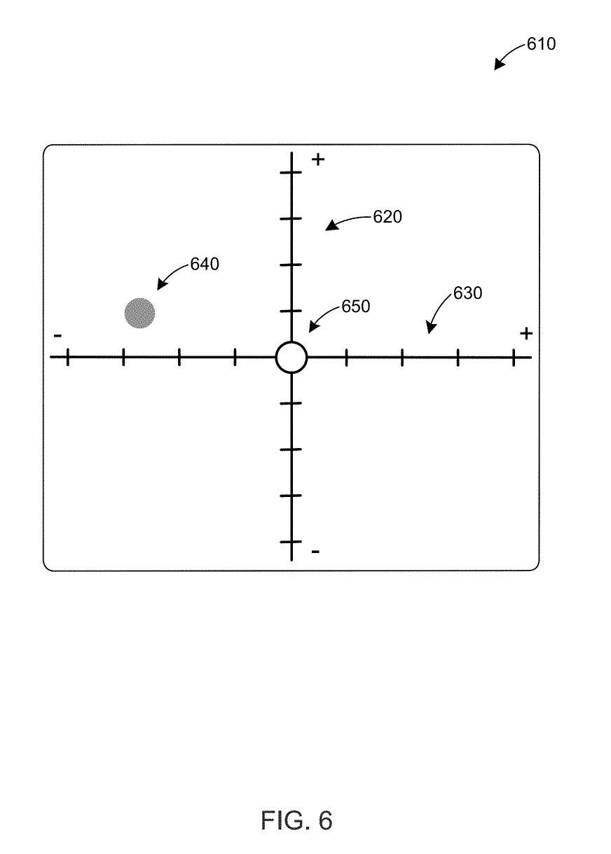

FIG. 6 is a front view of a display 610 that indicates the directions to rotate the device to align the second elevation and azimuthal angles 450, 575 with the first elevation and azimuthal angles 350, 375, respectively, according to one or more embodiments. The display 610 displays a vertical axis 620 to indicate the elevation angle error 460 and a horizontal axis 630 to indicate the azimuthal angle error 580. A circle 640 indicates the current value of the elevation angle error 460 and the current value of the azimuthal angle error 580. The position of circle 640 along the vertical and horizontal axes 620, 630 can be updated in real time or close to real time as the user changes the orientation of the device (e.g., as the user changes the elevation and/or azimuthal angle of the device).

A bullseye 650 indicates the orientation of the device at the first elevation and azimuthal angles 350, 375. When the circle 640 is located at the bullseye 640, the device and/or probe holder is oriented such that the second elevation and azimuthal angles 450, 575 are equal to (or substantially equal to) the first elevation and azimuthal angles 350, 375, respectively. When the circle 640 is located at the bullseye 640, the device can generate a visual or audible signal to indicate that the device is aligned, as discussed above. In some embodiments, the visual signal includes a graphic, a color change, a text box, or other visual element on display 610.

In general, if the elevation angle error 460 is positive, as indicated on vertical axis 620, the user needs to decrease the elevation angle of the device and/or probe holder (e.g., by rotating the device and/or probe holder downwardly) to align the current value of the second elevation angle 450 with the first elevation angle 250, which can be stored in the device's memory or in a memory operatively coupled to the device (e.g., in a removable memory or in a network-accessible memory). If the elevation angle error 460 is negative, as indicated on vertical axis 620, the user needs to increase the elevation angle of the device and/or probe holder (e.g., by rotating the device and/or probe holder and/or probe upwardly) to align the current value of the second elevation angle 450 with the first elevation angle 250. If the azimuthal angle error 580 is positive, as indicated on horizontal axis 630, the user needs to rotate the device and/or probe holder to the left to decrease the azimuthal angle of the device and/or probe holder to align the current value of the second azimuthal angle 575 with the first azimuthal angle 375, which can be stored in the device's memory or in a memory operatively coupled to the device (e.g., in a removable memory or in a network-accessible memory). If the azimuthal angle error 580 is negative, as indicated on horizontal axis 630, the user needs to rotate the device and/or probe holder to the right to increase the azimuthal angle of the device and/or probe holder to align the current value of the second azimuthal angle 575 with the first azimuthal angle 375.

As illustrated in FIG. 6, the circle 640 indicates that the device and/or probe holder has a positive elevation angle error 460 and a negative azimuthal angle error 580. Thus, the user needs to decrease the elevation angle (e.g., by rotating the device and/or probe holder and/or probe downwardly) and increase the azimuthal angle (e.g., by rotating the device and/or probe holder and/or probe to the right) to align the current value of the second elevation and azimuthal angles 450, 575 with the stored value of the first elevation and azimuthal angles 350, 375, respectively. After the device and/or probe holder and/or probe is aligned, the user may need to adjust the position of the probe holder so that the projected path 454 of the probe 420 passes through the mark.

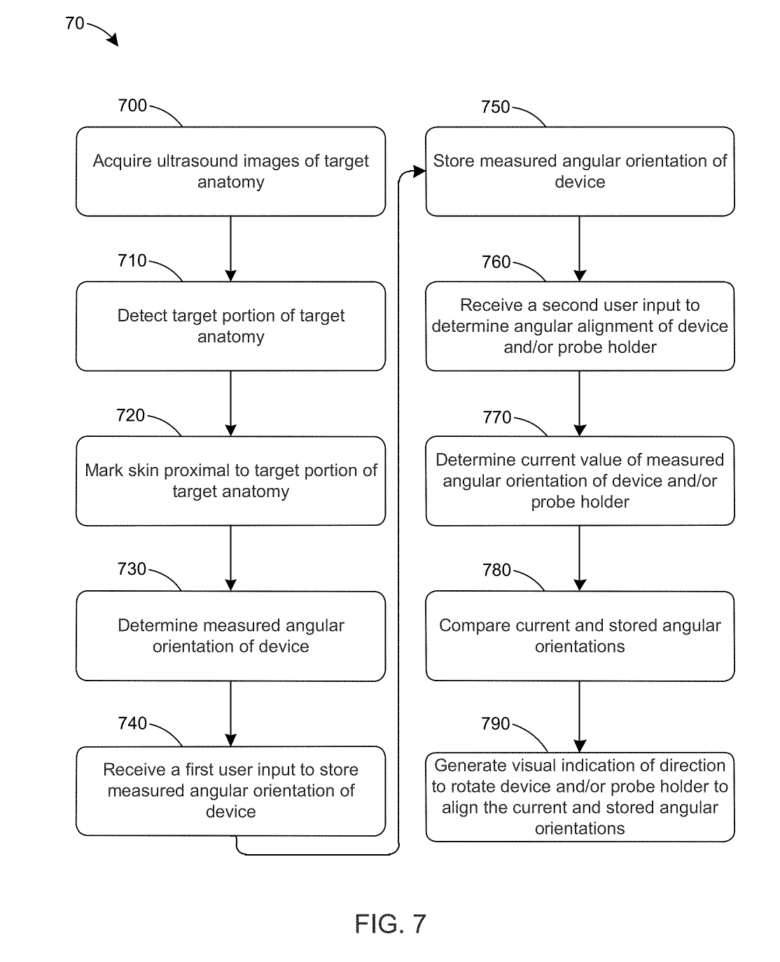

FIG. 7 is a flow chart 70 of a method for aligning an orientation of a probe according to one or more embodiments. Flow chart 70 can be performed using any of the devices described herein. In step 700, ultrasound images of target anatomy in a subject, such as a spine, are acquired with an ultrasound imaging device (e.g., device 200). The acquired images can be displayed on a display screen on the ultrasound imaging device or in electrical communication with the ultrasound imaging device (e.g., an external display). In step 710, a target portion of the target anatomy is detected. For example, the target epidural space of a spine can be detected by adjusting the position and/or angular orientation of the ultrasound imaging device, and by viewing the corresponding ultrasound images on the display screen. After the target portion of the anatomy is detected, in step 720 the skin proximal to the target portion of the target anatomy is marked with a marking unit while maintaining the desired angular orientation of the ultrasound imaging device. The marking unit can be included with or coupled to (e.g., detachably coupled to) the ultrasound imaging device.

In step 730, the angular orientation of the ultrasound imaging device is determined, for example, with an angle sensor disposed in the ultrasound imaging device. The angle sensor can include an accelerometer, a gyroscope, or other angle sensor, as discussed above. The angular orientation includes the elevation angle and the azimuthal angle of the ultrasound imaging device when the ultrasound imaging device is oriented to detect the target portion of the target anatomy. In step 740, a first user input is received by the ultrasound imaging device. A user can provide the first user input when the user wants to store the angular orientation of the ultrasound imaging device. For example, the user can provide the first user input when the ultrasound imaging device is oriented to detect the target portion of the target anatomy. The first user input can include an activation of a physical or virtual button or another user input device, as described above. In response to the first user input, in step 750 the ultrasound imaging device (e.g., a processor in the ultrasound imaging device) stores the current measured angular orientation (e.g., elevation and/or azimuthal angles) of the ultrasound imaging device in a non-transitory memory in or coupled to the ultrasound imaging device as a stored angular orientation.

Later (e.g., after prepping the site for a procedure), in step 760 the ultrasound imaging device receives a second user input to determine whether the ultrasound imaging device and/or probe holder is in angular alignment with the stored angular orientation. The second user input can include an activation of a physical or virtual button or another user input device, as described above. In step 770, the angle sensor determines the current value of the angular orientation of the ultrasound imaging device and/or probe holder and/or probe. In step 780, the ultrasound imaging device (e.g., the processor) compares the current and stored angular orientations. When the ultrasound imaging device and/or probe holder and/or probe is not in angular alignment with the stored angular orientation, in step 790 the ultrasound imaging device (e.g., the processor) generates a visual indication (e.g., on a display on the ultrasound imaging device) of the direction(s) to rotate the ultrasound imaging device to align the current and stored angular orientations. The visual indication can include a first direction to rotate the ultrasound imaging device and/or probe holder and/or probe to align the current elevation angle with the stored elevation angle and/or a second direction to rotate the ultrasound imaging device and/or probe holder and/or probe to align the current azimuthal angle with the stored azimuthal angle. When the ultrasound imaging device and/or probe holder is in angular alignment with the stored angular orientation, the ultrasound imaging device can generate a visual and/or audio signal to indicate such alignment, as discussed above.

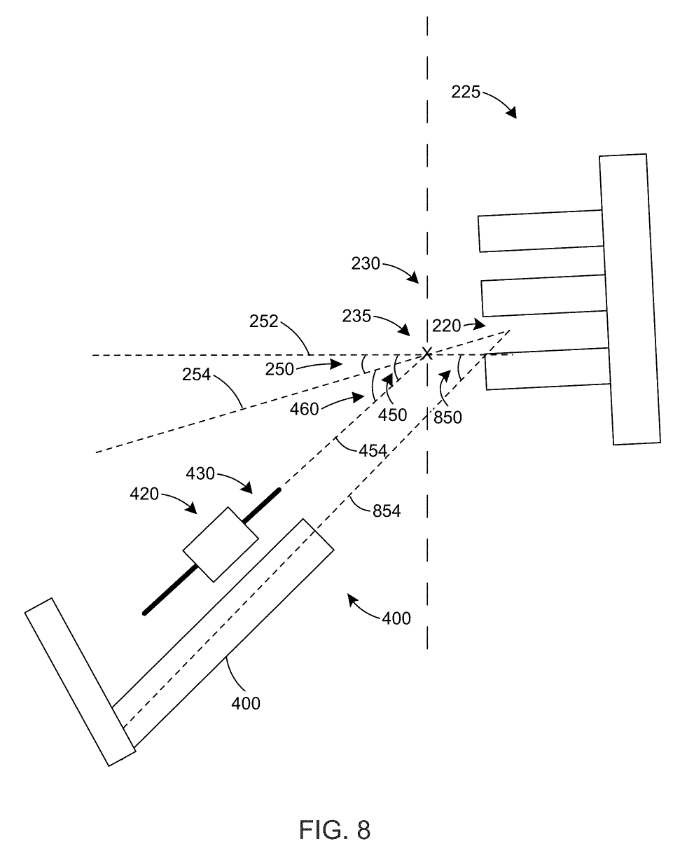

FIG. 8 is a side view of a misalignment of the second elevation angle 450 with respect to the first elevation angle 250 according to an alternative embodiment. In this embodiment, the probe guide 420 is separate from the device 400. The user holds the probe guide 420 and the device 400 so that their angular orientation is the same or substantially the same. When their angular orientation is the same or substantially the same, the projected path 454 of probe 430 is parallel to or substantially parallel to a line 854 that passes through the center of the housing 410 of the device 400. Since the projected path 454 and the line 854 are parallel to or substantially parallel to each other, the second elevation angle 450 of the projected path 454 of probe 430 is equal to or substantially equal to second elevation angle 850 of device 400, which is defined by the normal 252 to the skin surface 230 and line 854. This allows the user to indirectly determine and adjust the second elevation angle 450 of the projected probe path 454 by determining and adjusting the second elevation angle 850 of the device 400 while maintaining the parallel angular orientation of the probe guide 420 and the device 400. When the second elevation angle 850 is equal to or approximately equal to the first elevation angle 250, the second elevation angle 450 will also be equal to or approximately equal to the first elevation angle 250 provided that the angular orientation of the probe guide 420 and the device 400 are the same or substantially the same.

In an alternative embodiment, the angular orientation of the probe guide 420 is determined by another position and/or angular tracking system, such as one or more cameras or other optical tracking systems that measure the position and/or angular orientation of the probe guide 420. The user can manually adjust the elevation angle of the probe guide 420 so that its elevation angle, as measured by the foregoing position and/or angular tracking system, is the same as or substantially the same as the first elevation angle 250.

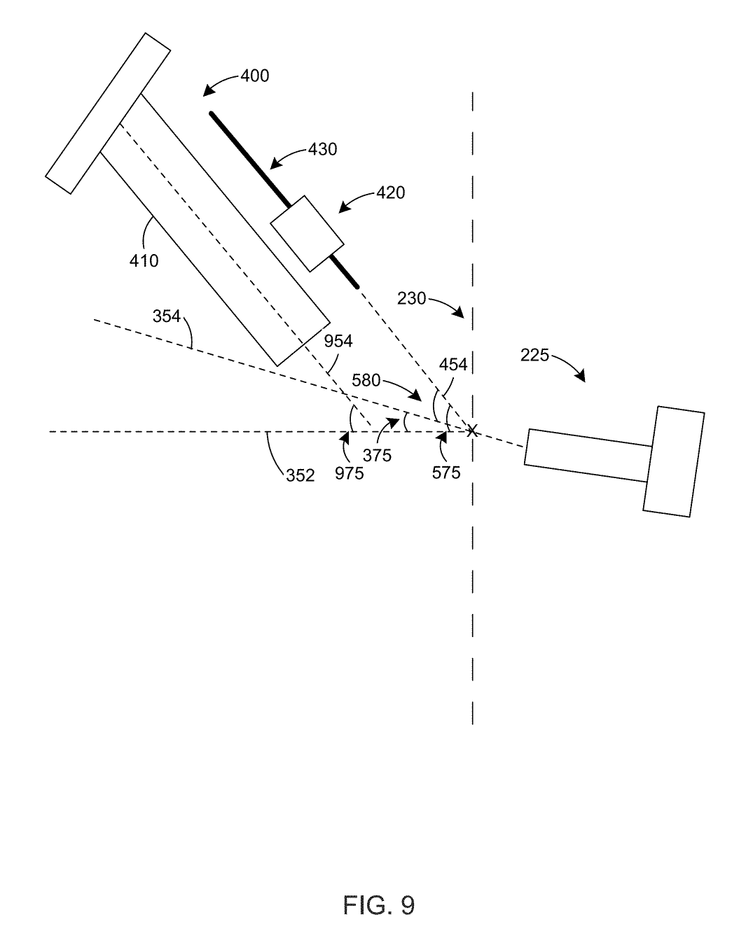

FIG. 9 is a top view of a misalignment of a second azimuthal angle 575 with respect to the first azimuthal angle 375 according to an alternative embodiment. In this embodiment, the probe guide 420 is separate from the device 400, as in FIG. 8. The user holds the probe guide 420 and the device 400 so that their angular orientation is the same or substantially the same. When their angular orientation is the same or substantially the same, the projected path 454 of probe 430 is parallel to or substantially parallel to a line 954 that passes through the center of the housing 410 of the device 400. Since the projected path 454 and the line 954 are parallel to or substantially parallel to each other, the second azimuthal angle 575 of the projected path 454 of probe 430 is equal to or substantially equal to the second azimuthal angle 975 of device 400, which is defined by the normal 252 to the skin surface 230 and line 954. This allows the user to indirectly determine and adjust the second azimuthal angle 575 of the projected probe path 454 by determining and adjusting the second azimuthal angle 975 of the device 400 while maintaining the parallel angular orientation of the probe guide 420 and the device 400. When the second azimuthal angle 975 is equal to or approximately equal to the first azimuthal angle 375, the second azimuthal angle 575 will also be equal to or approximately equal to the first azimuthal angle 375 provided that the angular orientation of the probe guide 420 and the device 400 are the same or substantially the same.

In an alternative embodiment, the angular orientation of the probe guide 420 is determined by another position and/or angular tracking system, such as one or more cameras or other optical tracking systems that measure the position and/or angular orientation of the probe guide 420. The user can manually adjust the azimuthal angle of the probe guide 420 so that its azimuthal angle, as measured by the foregoing position and/or angular tracking system, is the same as or substantially the same as the first azimuthal angle 375.

In an alternative embodiment, the probe guide or holder can itself contain angle-sensing components and/or a display to indicate angular positioning errors. In this case the first angles of orientation (azimuthal and/or elevational) can be obtained by the device (and transferred manually or automatically to the probe guide), or by the probe guide itself, if attached to the device. Next, during probe guidance, the device does not necessarily have to be used, as the guide can sense angular orientation and can provide feedback by a display, or other audio/visual indications. In some variations of this embodiment, the probe guide can use wireless communications to utilize a tablet, cellphone or other remote device as a display and/or indicator, for example using a Bluetooth-enabled iPhone or Android application to indicate the orientation feedback of FIG. 6.

The present invention should not be considered limited to the particular embodiments described above, but rather should be understood to cover all aspects of the invention as fairly set out in the attached claims. Various modifications, equivalent processes, as well as numerous structures to which the present invention may be applicable, will be apparent to those skilled in the art to which the present invention is directed upon review of the present disclosure. The claims are intended to cover such modifications and equivalents.

What is claimed is:

1. A handheld ultrasound imaging device comprising:
a housing;
an ultrasound imaging unit disposed in the housing proximal to a first surface of the housing;
an angle sensor disposed in the housing, the angle sensor outputting an angular orientation signal, the angular orientation signal corresponding to a measured angular orientation of the housing;
a display disposed on a second surface of the housing, the first and second surfaces opposing one another, the display including a graphical user interface;
said graphical user interface including a first graphical user interface element that generates a first GUI output signal in response to a first user input and a second graphical user interface element that generates a second GUI output signal in response to a second user input;
a memory disposed in the housing; and
a processor disposed in the housing, the processor in electrical communication with the display and the angle sensor, wherein a receipt of the first GUI output signal causes the processor to store the measured angular orientation in the memory as a stored angular orientation and a receipt of the second GUI output signal causes the processor to compare the stored angular orientation with a current value of the measured angular orientation.

2. The device of claim 1, wherein the receipt of the second GUI output signal causes the processor to calculate a direction to rotate the housing to align the current value of the measured angular orientation with the stored angular orientation.

3. The device of claim 1, further comprising a probe guide coupled to the housing, the probe guide having a predetermined path along which to insert a probe.

4. The device of claim 1, further comprising a marking unit coupled to the housing and configured to produce a mark on a surface of a target to be imaged.

5. The device of claim 1, wherein the processor is configured to generate a direction output signal that causes the display to graphically indicate, on the graphical user interface, an indication of the current angular orientation relative to the stored angular orientation.

6. The device of claim 5, wherein the direction output signal causes the display to graphically indicate, on the graphical user interface, a direction to rotate the probe guide.

7. The device of claim 6, wherein the direction output signal causes the display to graphically indicate a first direction to rotate the probe guide to adjust an elevation angle of a projected probe path of the probe.

8. The device of claim 6, wherein the direction output signal causes the display to graphically indicate a second direction to rotate the probe guide to adjust an azimuthal angle of a projected probe path of the probe.

9. The device of claim 8, wherein the direction output signal causes the display to graphically indicate a first direction to rotate the probe guide to adjust an elevation angle of the projected probe path of the probe such that the display simultaneously graphically indicates the first and second directions to rotate the probe guide.

10. The device of claim 1, wherein the processor is configured to generate an alignment signal when the current value of the measured angular orientation is substantially equal to the stored angular orientation.

11. The device of claim 10, wherein the alignment signal causes the display to graphically indicate, on the graphical user interface, that the current value of the measured angular orientation is substantially equal to the stored angular orientation.

12. The device of claim 10, wherein the alignment signal causes a speaker to generate an audible alert to indicate that the current value of the measured angular orientation is substantially equal to the stored angular orientation, the speaker disposed on or in the housing.

13. The device of claim 10, wherein the alignment signal causes an LED to emit a light to indicate that the current value of the measured angular orientation is substantially equal to the stored angular orientation, the LED disposed on the housing.

14. The device of claim 1, wherein the angle sensor includes an accelerometer or a gyroscope.

15. An ultrasound imaging method comprising:
in a probe guidance system comprising a processor disposed in a housing:
determining a measured angular orientation of the housing with an angle sensor disposed in the housing;
receiving a first user input to store the measured angular orientation;
in response to the first user input, storing the measured angular orientation as a stored angular orientation in a memory in electrical communication with the processor;
receiving a second user input to determine an angular alignment of a projected probe path of a probe disposed in the probe holder, the probe holder coupled to the housing; and
in response to the second user input, comparing a current measured angular orientation of the projected probe path with the stored angular orientation.

16. The method of claim 15, further comprising generating a visual indication of a direction to rotate the probe holder to align the current measured angular orientation with the stored angular orientation.

17. The method of claim 15, further comprising:
transmitting one or more ultrasound signals from one or more transducers in the probe guidance system;
obtaining ultrasound data generated based, at least in part, on one or more ultrasound signals from an imaged region of a subject; and
displaying an ultrasound image of a target anatomy in the subject based, at least in part, on the ultrasound data.

18. The method of claim 17, further comprising:
adjusting an angular orientation of the housing to align the housing with a target location in the target anatomy; and
marking a skin surface of the subject corresponding to the target location in the target anatomy.

19. The method of claim 18, wherein the target anatomy comprises a spine and the target location comprises an epidural space in the spine.

20. The method of claim 18, further comprising:
aligning the projected probe path of the probe with the mark on the skin surface; and
adjusting the angular orientation of the probe holder according to the visual indication so the current measured angular orientation is substantially aligned with the stored angular orientation.

21. The method of claim 20, further comprising inserting the probe into the skin surface along the projected probe path, the probe passing through the mark on the skin surface while the current measured angular orientation is substantially aligned with the stored angular orientation.

22. The method of claim 20, wherein the visual indication indicates a first direction to rotate the probe guide to adjust an elevation angle of the projected probe path.

23. The method of claim 20, wherein the visual indication indicates a second direction to rotate the probe guide to adjust an azimuthal angle of the projected probe path.

24. The method of claim 23, wherein the visual indication indicates a first direction to rotate the probe guide to adjust an elevation angle of the projected probe path such that the visual indication simultaneously indicates the first and second directions to rotate the probe guide.

25. The method of claim 20, further comprising generating a visual alignment indication that indicates that the current measured angular orientation is substantially aligned with the stored angular orientation.
